(19) United States
(12) Patent Application Publication
(10) Pub. No.: US 2009/0088684 A1
(43) Pub. Date: Apr. 2, 2009

(54) FREEZER BAG

(76) Inventor: Peter Rohrig, Wien (AT)

Correspondence Address: LADAS & PARRY LLP, 224 SOUTH MICHIGAN AVENUE, SUITE 1600, CHICAGO, IL, 60604, US

(21) Appl. No.: 12/278,991

(22) PCT Filed: Feb. 9, 2007
(86) PCT No.: PCT/AT2007/000067

§ 371 Date: Aug. 11, 2008

(30) Foreign Application Priority Data
Feb. 10, 2006 (AT) A 211/2006

(51) Int. Cl. A61M 1/06

(52) U.S. Cl. 604/74

(57) ABSTRACT

A breast milk freezer bag for receiving and conserving breast milk. The freezer bag includes a filling opening which has a collar which extends over the upper edge of the breast milk freezer bag and which is used to secure the breast milk freezer bag to a breast pump. At least two receiving units which are connected in a communicating manner, respectively, to at least one adjacent receiving unit by at least one connection channel are provided such that the breast milk in the receiving units can, in the frozen state, be distributed in portions which correspond to the receiving volume of the receiving units and in the defrosted state, can be administered in a portioned manner.

The invention relates to a breast milk freezer bag for receiving and storing breast milk, including a filling opening, said filling opening having a collar extending over the upper edge of the breast milk freezer bag for securing the breast milk freezer bag to a breast pump.

Breast milk freezer bags are containers for breast milk, which are made of flexible, thin-walled materials and intended for filling with fresh breast milk and cooling or deep-freezing of the latter.

Breast milk freezer bags of this type having but a single reception unit for receiving breast milk are known from CA 2 300 297 A1, JP 08 198 297 A and JP 2004-010155A. Those freezer bags, after having been filled with milk, are deep-frozen and, for feeding a baby, are introduced into a feeding bottle either in the frozen state or in a thawed state, comprising a special opening and closing mechanism to enable the tight closure and easy opening of the bag.

Other breast milk freezer bags having but a single reception unit are known from U.S. Pat. No. 6,899,239 B1 and US 2002/0156419 A1. Those freezer bags can be connected with a breast pump, to which end they are equipped with a specially designed upper edge or a specially designed filling opening so as to enable the breast milk pumped off by the aid of the breast pump to be directly filled into the reception unit. The breast milk freezer bag according to U.S. Pat. No. 6,899,239 B1, moreover, exhibits a spring-loaded latch to close the reception unit or bag for receiving the pumped-off breast milk, if the milk contained therein is not fed completely, so as to store the remaining residue of the breast milk.

From JP 2001-299905 A, a breast pump including a feeding bottle and a milk storage container especially devised for that feeding bottle are known. That special storage container too has but one reception area for the breast milk, the filling opening of the container being conically shaped for connection with the breast pump.

All of those known breast milk freezer bags involve the drawback that they are exclusively provided for receiving larger amounts of milk. If, after the deep-frozen storage, only a smaller amount of milk is required, the entire bag will have to be thawed or heated. The milk that is not required will remain in the bag unused and have to be disposed of or consumed within a very short time, since it would otherwise be unusable for a baby for hygienic and health reasons. As a result, such breast milk freezer bags are, in particular, disadvantageous for mothers who have only relatively small quantities of breast milk, since a substantial part of the pumped-off milk has to be discarded unused after thawing.

A disposable bag of another type for receiving milk powder and water is known from U.S. Pat. No. 2,885,104 A. That disposable bag is subdivided into two sections separated by a weld and provided for receiving water, on the one hand, and milk powder, on the other hand. The two sections, which are superimposed in the operating position, can be transformed into a unitary section by means of a strip co-molded into the separation seam during the production of the insert. To this end, the lower end of the strip, which is guided through the partially open bottom of the baby bottle, is pulled. This causes the separation seam of the two sections to be destroyed or separated such that the water contained in the upper section will enter the lower section containing the milk powder.

Furthermore, freezer bags of different types, which are subdivided into several reception units, are already known in principle. Such freezer bags as are, for instance, disclosed in U.S. Pat. No. 4,783,042 A, DK 104 681 A and U.S. Pat. No. 4,223,043 A, are, however, not suitable for connection with breast pumps and, apart from that, are merely provided for the preparation of deep-frozen popsicles or frozen candies to be licked or consumed one at a time in the frozen state.

By contrast, it is the object of the invention to provide a breast milk freezer bag of the initially defined kind, which allows different amounts of breast milk to be readily made available according to demand without having to thaw the total breast milk contained in the breast milk freezer bag.

The breast milk freezer bag according to the invention is characterized in that at least two reception units are provided, wherein the reception units are each connected with at least one adjacent reception unit in a communicating manner via at least one connection channel such that the breast milk received in the reception units is dividable into portions in the frozen state as a function of the reception volumes of the reception units and feedable in portions in the thawed state.

The subdivision into at least two reception units merely connected via at least one connection channel allows the stored breast milk to be divided into smaller portions depending on the reception volumes of the reception units. The frozen breast milk portions, which are merely connected by the connection channel, can thereby be separated from one another in a simple manner, for instance, by pressing a portion of deep-frozen breast milk contained in a reception unit out of the reception unit. Thus, all of the milk contained in the breast milk freezer bag can actually be fed to a baby in portions and no thawed milk has to be disposed of unused.

If the reception units are separable from one another along a plane dividing the connection channel, it is feasible to separate the reception units as a function of the amount of milk required, i.e. one or more reception units, from the remaining reception units in the deep-frozen state, e.g. by tearing or cutting, so as to enable in a simple manner to thaw only that amount of milk which is actually required.

With a view to ensuring the simple manufacture of mutually separated reception units that are interconnected via a connection channel, it is beneficial if the breast milk freezer bag is made of two plastic surfaces which are planarly welded together while leaving free the filling opening, the reception units and the connection channels. The plastic surfaces are preferably flexible, transparent or colored, yet advantageously substantially translucent, thin-layer sheets which are suitable for the hygienic and, in particular, bacteria-free storage and deep-freezing of breast milk. In this respect, it is conceivable to provide a plurality of breast milk freezer bags that are welded together via their upper and lower edges, viewed in the filling position, in the form of a strand or strip for their distribution, with the individual breast milk freezer bag being configured to be readily separable from the following breast milk freezer bag through a perforation.

In order to enable the simple separation of the reception units of the breast milk freezer bag, e.g. by cutting, it is advantageous if the reception units are interconnected by connection portions which are planarly welded while leaving free the connection channels.

In order to enable the convenient separation of a reception unit from its adjacent reception unit, it is advantageous if the connection portions have perforations. The separation will be further simplified if the connection portions each have a separation notch in at least one edge portion.

With a view to providing a reliable transfer between the reception units during the filling of the breast milk freezer bags with breast milk, and with a view to enabling the simple separation of the individual adjacent reception units, it is suitable that the reception units are arranged one above the other in the filling position of the breast milk freezer bag.

In order to utilize the reception volume of the breast milk freezer bag as well as possible, it is favorable if the reception units are designed in a substantially cylindrical manner.

In this context, it is advantageous for as efficient a utilization as possible of the available reception volume, if the base and top surfaces of the substantially cylindrical reception units are arranged in parallel.

In order to enable the rapid and efficient transfer between the reception units during the filling of milk, it is favorable if the connection channels between the reception units are arranged in the longitudinal axis of the substantially cylindrical breast milk freezer bag.

In order for the milk to be filled into the breast milk freezer bag without difficulty while being uniformly distributed within the reception units, it is suitable that the filling opening runs from the upper edge of the freezer bag into the first or uppermost reception unit, viewed from the upper edge of the freezer bag.

By providing an appropriately configured collar on the upper edge of the breast milk freezer bag, it is feasible in a simple manner to fasten the freezer bag to a jacket-like housing of a breast pump by the aid of a cap in the manner of a union nut. If the filling opening is designed in a funnel-shaped manner, the pumped-off breast milk will reliably flow into a first reception unit. The funnel-like shape can be differently devised as a function of the type of breast pump to be used. It may have differently large dimensions in its widest diameter near the upper edge, viewed in the filling position of the breast milk freezer bag. A funnel-shaped connection socket of a breast pump for the milk outlet from the pump can, thus, be directly contacted with the filling opening of the breast milk freezer bag.

With a view to ensuring efficient utilization of the reception volume of the breast milk freezer bag, it is alternatively also possible that laterally adjacently arranged reception units are provided, whose longitudinal axes are substantially vertical in the filling position. In this respect, it is advantageous, in order to enable the transfer of breast milk between the reception units by the shortest way, if the connection channels between the reception units are arranged perpendicular to the longitudinal axes of the reception units.

In order to enable the uniform filling of the adjacently arranged reception units with breast milk, it is beneficial if the filling opening communicates with all of the laterally adjacently arranged reception units.

It has turned out that reception volumes of the reception units of from about 10 ml to about 50 ml, preferably about 20 ml to about 40 ml, in particular about 30 ml, enable the suitable apportioning of milk meals for a baby.

For the use of milk from the breast milk freezer bag, it is beneficial to provide at least one labeling surface on one of the outer sides of the freezer bag for indicating the date and/or hour of filling of the milk on the breast milk freezer bag.

To ensure simple manufacture and durability, it is advantageous if the breast milk freezer bag is made of a thermoplastic material.

In the following, the invention will be explained in more detail by way of exemplary embodiments illustrated in the drawings. Therein:

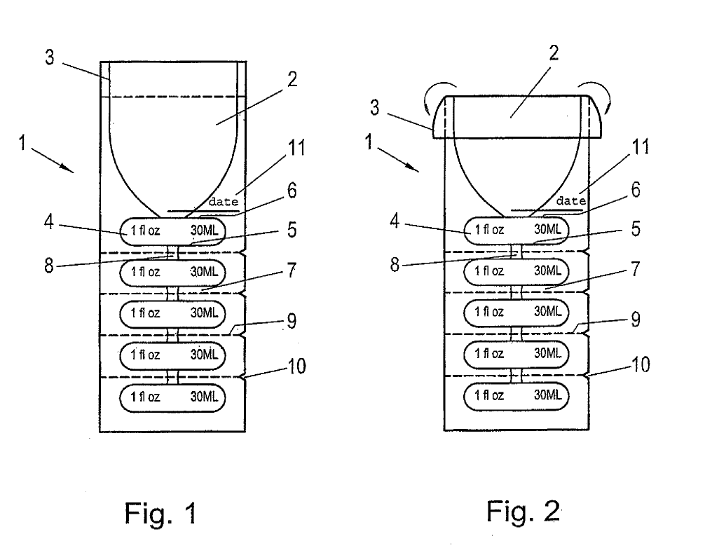

FIGS. 1 and 2 each illustrate a breast milk freezer bag;

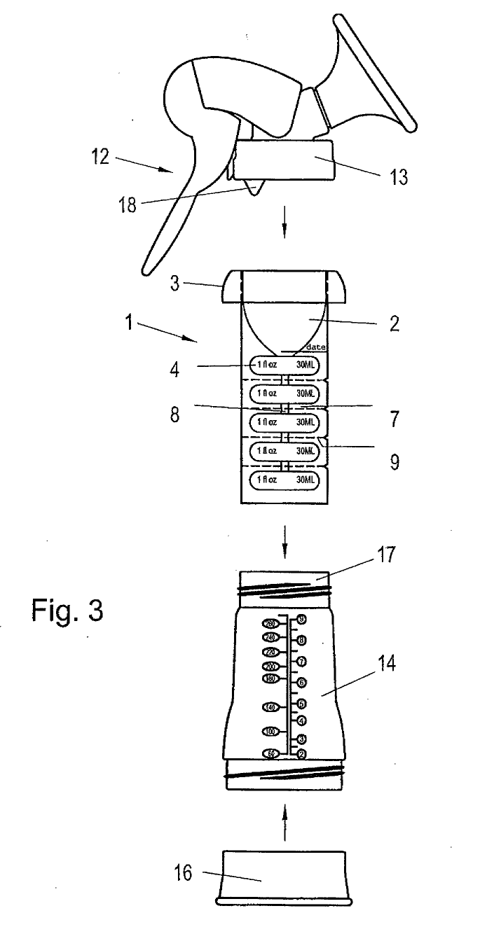

FIG. 3 is an explosive view of a breast pump arrangement including a breast milk freezer bag according to FIG. 1 and FIG. 2;

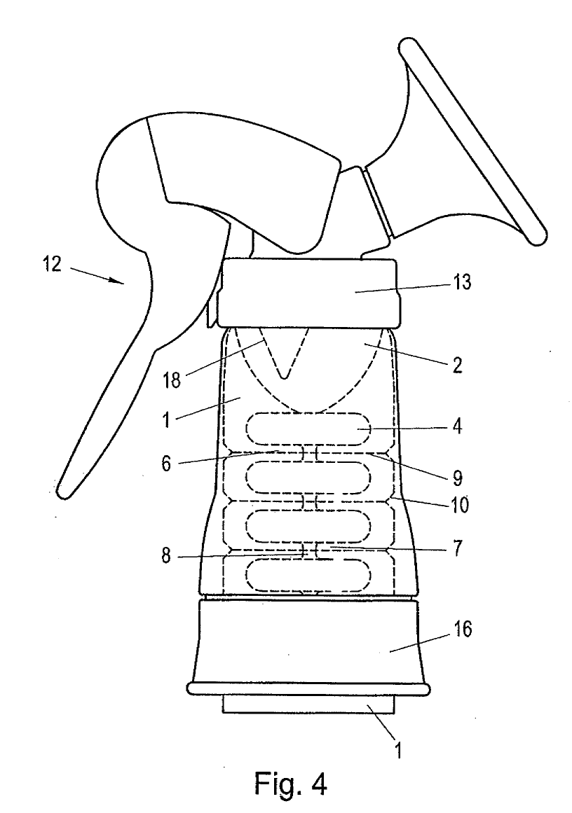

FIG. 4 is an assembled view of a breast pump arrangement according to FIG. 3;

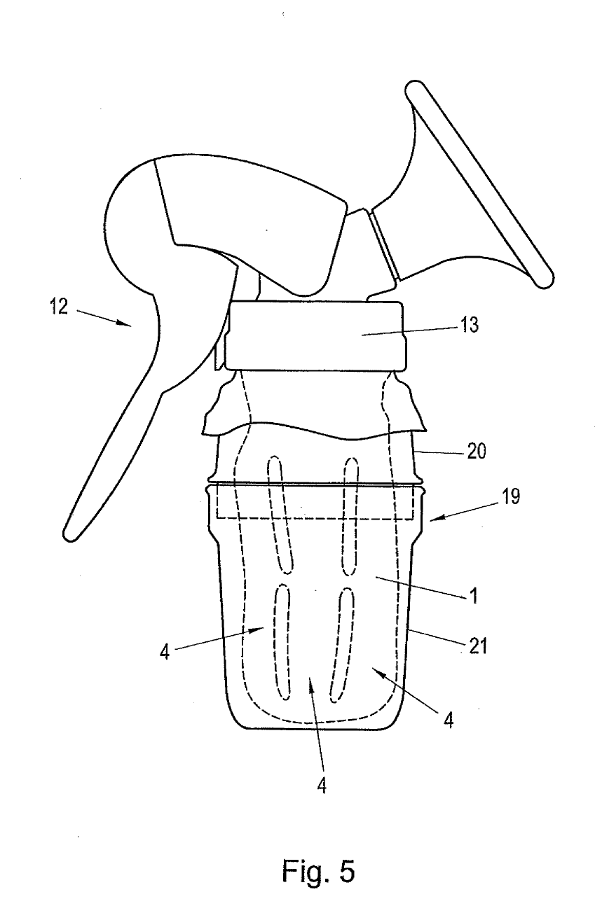

FIG. 5 is an assembled view of the breast pump including an alternative exemplary embodiment of the breast milk freezer bag;

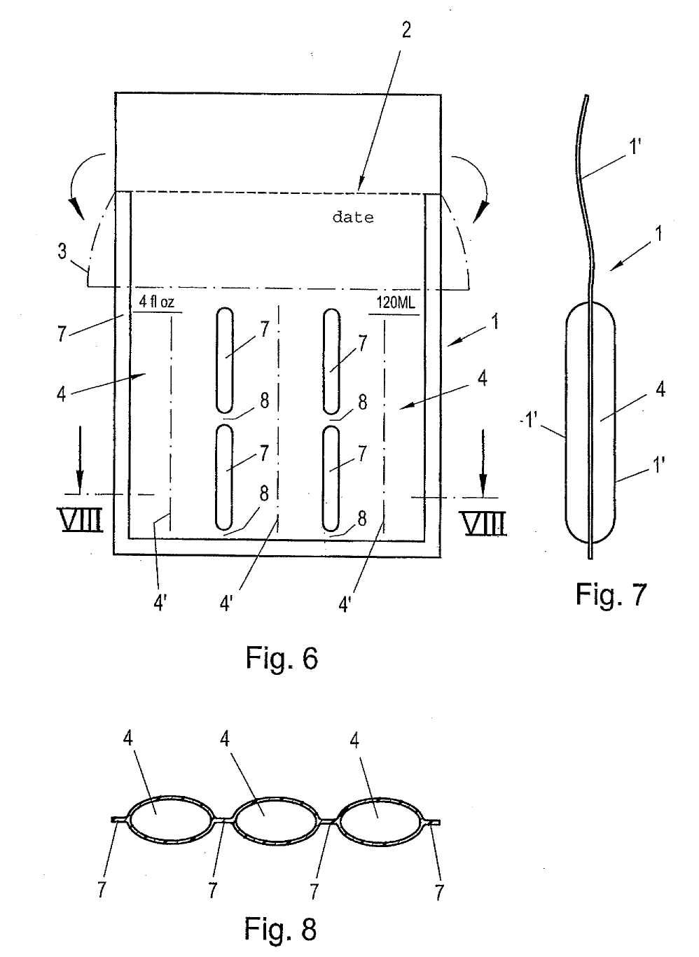

FIG. 6 is a view of a breast milk freezer bag including laterally adjacently arranged reception units;

FIG. 7 is a side view of the breast milk freezer bag according to FIG. 6; and

FIG. 8 is a section along line VIII-VIII of FIG. 6.

FIG. 1 depicts a breast milk freezer bag 1 according to the invention for receiving and storing breast milk, which comprises several reception units 4. The breast milk freezer bag 1 has a substantially cylindrical shape and is formed by two partially welded plastic sheets of, e.g., thermoplastic material.

To fill the breast milk freezer bag 1, a central filling opening 2 having a funnel-shaped form and a collar 3 extending along the upper edge of the breast milk freezer bag 1 are provided in the upper portion of the breast milk freezer bag 1. As is, in particular, apparent from FIGS. 2 and 3, the collar 3 can be turned inside out so as to enable the simple fixation of the breast milk freezer bag 1 to a breast pump for pumping off breast milk.

The filling opening 2, by its lower end, runs into a first reception unit 4. The reception unit 4, which has a base surface 5 and a top surface 6, is substantially cylindrically designed, with its longitudinal axis extending in parallel with the longitudinal axis of the substantially cylindrical freezer bag 1. In total, the breast milk freezer bag 1 comprises five reception units 4, which are arranged one above the other. The reception units 4, which are arranged in a spaced-apart relationship, are located centrally in respect to the breast milk freezer bag 1 and interconnected by connection portions 7. Within the connection portions 7, the plastic sheets are welded together in a planar fashion, with the connection channels 8 being left unwelded. The connection channels 8 likewise extend in the center of the base surface 5 of a reception unit 4 as far as to the top surface 6 of the reception unit 4 arranged therebelow, so that the individual reception units 4 are interconnected so as to communicate along a vertical line.

The connection portions 7 are readily separable even in the deep-frozen state of the breast milk along perforations 9 provided therefor, so that individual reception units 4 can be separated from the remaining reception units 4 and separately thawed for consumption. In this respect, it is, of course, also conceivable that the connection portions 7 can be separated by using scissors or a Stanley knife, so that perforations 9 are not necessarily required. To further promote the separation of the reception units 4 from one another, separation notches 10 are provided on the lateral edge or edge portion of the breast milk freezer bag 1, at the end of the perforations 9.

The reception units 4 have markings on their external surfaces to indicate the volumes of the reception units 4. In the exemplary embodiment illustrated, all of the reception units 4 have identical reception volumes, yet it is, of course, also possible to provide reception units 4 having different reception volumes. Furthermore, a labeling surface 11 is provided near the lower end of the filling opening 2 for indicating the respective milk filling date on the breast milk freezer bag 1.

To connect the breast milk freezer bag 1 to a breast pump 12, the collar 3 extending from the upper edge of the filling opening 2 is initially folded down or turned inside out. As illustrated in FIG. 3, the breast pump 12 comprises a union cap 13 with a female thread to be screwed on a jacket 14. In this respect, a jacket 14 as used for a baby bottle can be employed as well. For feeding the pumped-off breast milk, the same jacket 14 can, thus, be used after having screwed on a bottom cap and attached a teat, whereby the overall number of parts of the devices for pumping and feeding will be reduced. A ring 16 of nonskid material can be screwed onto the foot of the jacket 14 for the stability of the breast pump 12.

To connect the breast milk freezer bag 1 to the breast pump 12, the collar 3 of the filling opening 2 of the breast milk freezer bag 1 is, at first, folded outwardly (cf. FIG. 2), and after this the breast milk freezer bag 1 is introduced into the jacket 14 so as to encompass the upper edge 17 of the jacket 14. Subsequently, the union cap 13 of the milk pump 12 is screwed onto the jacket 14 such that the breast milk freezer bag 1 is secured to the edge 17 of the jacket 14 by its collar 3 and cannot be detached unintentionally.

As is apparent from FIG. 4, a conically shaped outlet 18 of the breast pump 12 projects into the funnel-shaped filling opening 2 of the breast milk freezer bag 1 in the assembled state. When pumping milk off while using the breast pump 12, the pumped-off milk is thus directly introduced into the filling opening 2 from the outlet 18 of the breast pump 12, and into the uppermost reception unit 4 via the funnel-shaped filling opening 2.

Via the vertically arranged connection channels 8, the pumped-off milk flows directly from the first reception unit 4 into the reception units 4 located therebelow.

From FIG. 4, it is further apparent that the lower portion of the breast milk freezer bag 1 may project out through the central opening provided in the ring 16. By providing a jacket 14 with an open bottom, the breast milk freezer bag 1 in the filled state may even have a larger longitudinal extension than the jacket 14 such that the use of a breast milk freezer bag having more than the five reception units 4 illustrated can, of course, be envisaged. The number of reception units 4 of the breast milk freezer bag 1 may consequently be varied as a function of the desired reception quantity of the freezer bag 1.

FIG. 5 depicts an alternative exemplary embodiment of the breast milk freezer bag 1, in which laterally adjacent reception units 4 are provided in the filling position (cf., in particular, also FIGS. 6 and 8). Moreover, the downwardly open jacket 14 is replaced with a closed container 19 comprised of an upper part 20, onto which the union cap 13 of the milk pump 12 is screwed, and a lower part 21, said upper and lower parts 20, 21 being connected with each other by a screw connection.

As is apparent from FIGS. 6 to 8, the breast milk freezer bag 1 comprises several reception units 4, whose longitudinal axes 4′ are substantially vertically arranged in the filling position. The breast milk freezer bag 1 in this case is substantially formed by two plastic sheets 1′ which are welded together in a planar fashion in the connection portions 7. In order to enable the transfer of breast milk in the liquid state between the reception units 4, the connection portions 7 are interrupted by connection channels 8 between the reception units 4. As is further apparent from FIG. 6, the breast milk freezer bag 1 has a filling opening 2 simultaneously communicating with all of the laterally adjacent reception units 4.

In addition, the alternative breast milk freezer bag 1 represented in FIGS. 5 to 8, like the exemplary embodiment according to FIGS. 1 to 4, comprises a collar 3 extending about the upper edge of the breast milk freezer bag 1 and capable of being turned about the upper portion 20 of the container 19 so as to reliably fix the breast milk freezer bag 1 within the container 19 by the aid of the union cap 13 of the milk pump 12.

Once the reception units 4 of the breast milk freezer bag 1 are filled with pumped-off milk, the breast milk freezer bag 1 can be removed from the breast pump 12 and closed in any suitable manner. The breast milk freezer bag 1 can be closed by a rubber ring, or the user ties a knot in the region of the filling opening 2, whereupon the freezer bag 1 can be stored deep-frozen in a tightly closed manner. When using a partial amount of the pumped-off milk, one or more reception units 4 of the breast milk freezer bag 1 are separated according to demand from the breast milk freezer bag 1 in the frozen state in the region of their connection portions 7, optionally via the perforations 9 and separation notches 10 (in FIG. 4 on both edge portions of the freezer bag 1). It is, however, also possible to press the frozen milk portions in the reception units 4 out of the plastic sheet of the breast milk freezer bag 1—without previous separation of a reception unit 4—and introduce the milk into a baby bottle for its use in order to thaw or heat it for feeding. The deep-frozen breast milk still contained in the breast milk freezer bag 1 will thus remain substantially protected relative to the environment by the freezer bag 1 (apart from the open connection channel) so as to safeguard hygienic storage.

1-18. (canceled)

19. A breast milk freezer bag for receiving and storing breast milk, including a filling opening, said filling opening having a collar extending over the upper edge of the breast milk freezer bag and capable of being folded outwardly for securing the breast milk freezer bag to a breast pump, wherein at least two reception units are provided, whose reception volumes each comprise from about 10 ml to 50 ml, wherein the reception units are each connected with at least one adjacent reception unit in a communicating manner via at least one connection channel such that the breast milk received in the reception units is dividable into portions in the frozen state as a function of the reception volumes of the reception units and feedable in portions in the thawed state.

20. A breast milk freezer bag according to claim 19, wherein the reception units are separable from one another along a plane dividing the connection channel.

21. A breast milk freezer bag according to claim 20, wherein the breast milk freezer bag is made of two plastic surfaces which are planarly welded together while leaving free the filling openings, the reception units and the connection channels.

22. A breast milk freezer bag according to claim 21, wherein the reception units are interconnected by connection portions which are planarly welded while leaving free the connection channels.

23. A breast milk freezer bag according to claim 22, wherein the connection portions have perforations.

24. A breast milk freezer bag according to claim 19, wherein the connection portions each have a separation notch in at least one edge portion.

25. A breast milk freezer bag according to claim 19, wherein the reception units are arranged one above the other in the filling position of the breast milk freezer bag.

26. A breast milk freezer bag according to claim 25, wherein the reception units are designed in a substantially cylindrical manner.

27. A breast milk freezer bag according to claim 26, wherein the base and top surfaces of the substantially cylindrical reception units are arranged in parallel.

28. A breast milk freezer bag according to claim 25, wherein the connection channels between the reception units are arranged in the longitudinal axis of the substantially cylindrical breast milk freezer bag.

29. A breast milk freezer bag according to claim 25, wherein the filling opening runs from the upper edge of the breast milk freezer bag into the first or uppermost reception unit, viewed from the upper edge of the breast milk freezer bag.

30. A breast milk freezer bag according to claim 25, wherein the filling opening is designed in a funnel-shaped manner.

31. A breast milk freezer bag according to claim 19, wherein laterally adjacently arranged reception units are provided, whose longitudinal axes are substantially vertical in the filling position.

32. A breast milk freezer bag according to claim 31, wherein the connection channels between the reception units are arranged perpendicular to the longitudinal axes of the reception units.

33. A breast milk freezer bag according to claim 31, wherein the filling opening communicates with all of the laterally adjacently arranged reception units.

34. A breast milk freezer bag according to claim 19, wherein the reception volumes of the reception units comprise from about 20 ml to about 40 ml.

35. A breast milk freezer bag according to claim 19, wherein at least one labeling surface is provided on one of the outer sides of the freezer bag.

36. A breast milk freezer bag according to claim 19, wherein the breast milk freezer bag is made of a thermoplastic material.
